(19) United States
(12) Patent Application Publication
(10) Pub. No.: US 2010/0130942 A1
(43) Pub. Date: May 27, 2010

(54) Device for securing intravenous needles to treatment sites

(76) Inventor: Shirley Ann Rose, Naples, FL (US)

Correspondence Address: Isaac A. Angres, Suite 304B, 2001 Jefferson Davis Highway, Arlington, VA, 22202, US

(21) Appl. No.: 12/591,527

(22) Filed: Nov. 23, 2009

Related U.S. Application Data
(62) Division of application No. 11/341,163, filed on Jan. 27, 2006, now abandoned.

(51) Int. Cl. A61M 5/32

(52) U.S. Cl. 604/179

(57) ABSTRACT

The invention is directed to a system and device for securing one or more intravenous needles or cannulas to a patient in a treatment site such as on an arm, hand or leg. The device includes a semi-rigid but flexible pad that is placed in a location which is opposite of the treatment site, such as on the opposite side of an arm, hand or leg. The pad has straps emanating from it which extend around the patient and while doing so cross each other to trap a needle or cannula stabilizing feature and then continue around the patient and back to the pad. The pad has a first part of a hook and loop fastening system thereon, while the straps have the second part of the hook and loop fastening system thereon so that when engaged they will securely hold the straps in place. The pads may take on different shapes such as rectangular, oval or triangular shape to be able to be placed at different locations on the patient.

### CROSS REFERENCE TO RELATED APPLICATIONS

(none)

STATEMENT REGARDING FED SPONSORED R & D

(none)

### FIELD OF THE INVENTION

The present invention relates generally to medical devices and particularly to a device for securing intravenous needles, cannulas and tubing to treatment sites including arterial-venous fistula sites as used in dialysis treatment.

### BACKGROUND OF THE INVENTION

Intravenous needles and cannulas, also referred to as venous access devices, must he secured to patients to prevent dislodgment during medical treatment. The term needle will be used herein to refer to both needles and cannulas. The location of the needle insertion point or points on a patient vary with the type of medical treatment and the location of a suitable vein. Such locations include for example an upper arm, a surface on the outside surface of a hand, a wrist of a hand, a lower arm or an inside of an elbow of an arm. In dialysis treatment, a special site is typically created to provide for a strengthened vein, most commonly in the forearm and less frequently in the leg, to repeatedly accept two large needles and provide for sufficient flow of blood. The special site is created by joining a vein and an artery and is called a fistula and receives two arterial-venous (AV) fistula needles. In any of the above mentioned locations, it is very difficult to keep the needle secured to operate satisfactorily. In dialysis treatment, where the treatment is prolonged, repeated frequently, and two large needles are used, the problem of securing the needles is exasperated.

It is of the utmost importance that the needle or needles be kept under surveillance to check for proper operation and potential dislodgment or leaks of fluid. The most prevalent method to secure needles is to apply adhesive bandages and or tape to keep the needles in place. Unfortunately, bandages and tape can frequently obscure the view of the needle insertion point and needle assembly. Furthermore, many patients cannot tolerate prolonged or repeated applications of adhesive bandages and tape to their skin and have or develop an allergic reaction to adhesive bandages and tape. It has been known that patients who undergo dialysis treatments several times a week for a prolonged period of time can exhibit blue and black spots on their skin because of such an allergy to the adhesive and the continuous application thereof. Other solutions such as arm boards with straps around the location of the needle to keep it in place are not satisfactory because they severely restrict the movement of the patient. What is needed is a device that secures one or more intravenous needles, such as two arterial-venous fistula needles, securely in place, provides for observance of the treatment location, allows for some movement of the patient and does not require adhesive bandages.

### SUMMARY OF THE INVENTION

The invention at hand solves the above noted problems. In the securement of the needles or a needle in either a dialysis or other intravenous (IV) procedure, no adhesives arc used. Once a needle is placed within a vein, the needle is kept in place by straps that cross each other in that particular location. The straps are derived from and are fixed to a pad that is placed on an opposite side of the limb, hand or other part of the patient's body to which the needle is located. As the straps are moved around the location of the needle, they will cross that location and will return to their origin of the other side of the pad and will be fastened thereto. More than one strap will cross each other at the needle to thereby securely hold the needle in place at that location. A first strap can be used to apply a first pressure to secure the needle. A second strap can be used to apply a second pressure to further secure the needle.

### BRIEF DESCRIPTION OF THE DRAWINGS

FIG. 1 shows a double needle, such as two arterial-venous fistula needles, secured on an inside of an arm;

FIG. 2 shows the location of a pad on the outside of an arm from which straps will come forward to cross a needle;

FIG. 3 illustrates a typical pad, such as that depicted in FIG. 2, having straps thereon and how they are fastened thereto;

FIG. 4 shows a pad having straps thereon to be used on the inside of a hand;

FIG. 5 shows a needle secured on the outside surface of a hand;

FIG. 6 shows the pad of FIG. 4 as it is applied to the inside of a hand including the straps as they are fastened;

FIG. 7 illustrates a pad having straps thereon that is to be used on top of the wrist of a hand;

FIG. 8 illustrates the pad of FIG. 7 to show how the straps will wrap around the hand;

FIG. 9 illustrates a needle secured to the inside wrist of a hand;

FIG. 9a illustrates a needle secured to the inside wrist of a hand in an alternate location to that depicted in FIG. 9;

FIG. 10 shows a pad having straps thereon to be used on an elbow of an arm;

FIG. 11 shows a needle secured in the inside of an elbow;

FIG. 12 illustrates the pad of FIG. 10 as it holds the needle of FIG. 11 in place.

### DETAILED DESCRIPTION OF THE INVENTION

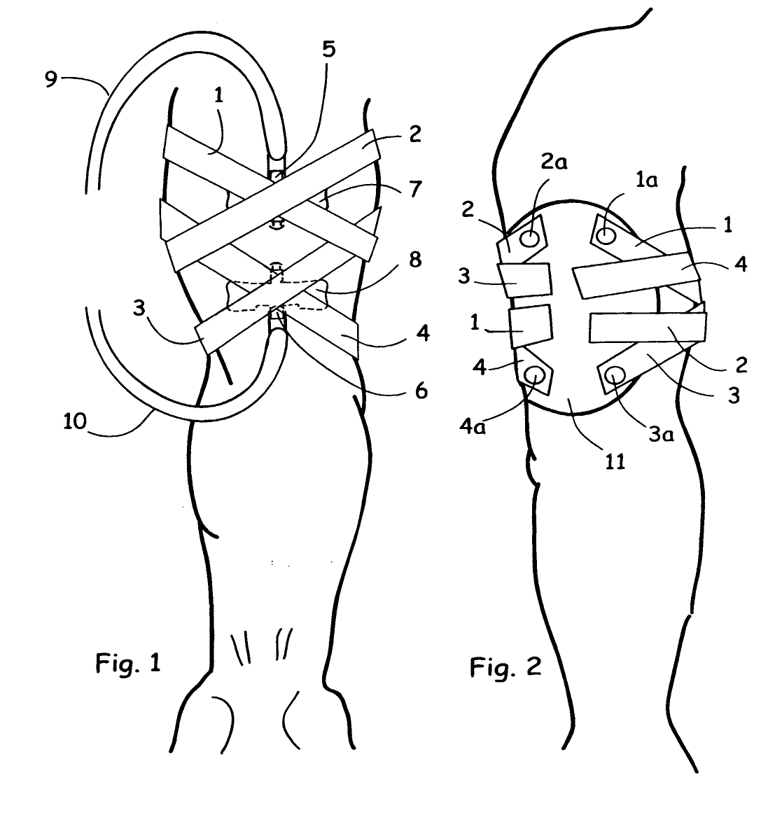

FIGS. 1 and 2 illustrate how the present invention is applied to an inner surface of an upper arm of a patient to secure two intravenous (IV) needles 5 and 6, such as two arterial-venous (AV) fistula needles. Needles 5 and 6 respectively have tubes 9 and 10 and tabs, also called wings, 7 and 8 attached thereto to form needle assemblies also referred to herein as needle assemblies 9 and 10, respectively. The embodiment depicted in FIGS. 1 and 2 employs four straps 1, 2, 3 and 4 to hold the two needles in place. In the orientation shown in FIGS. 1 and 2, straps 1 and 2 are upper straps and straps 3 and 4 are lower straps. Strap 1 is fastened to an oval pad 11 by way of a grommet 1a. Grommet 1a should be fastened in such a way so that the strap 1 can rotate relative to the pad 11 and can follow the angle of direction to which the strap 1 is forced when it is applied thereby preventing kinks. This is also true for grommets 2a, 3a and 4a which fasten straps 2, 3 and 4 to the pad 11 respectively. The pad 11 is applied to the outer arm if the locations if the needles 5 and 6 are applied to the inner arm. In this application, the upper straps 1 and 2 cross each other over the upper needle 5 and return to the pad 11 where both of the straps are fastened to the pad 11 by way of the well known hook and loop fastening system, VELCRO™. This is equally true for the lower straps 3 and 4 which cross each other over the lower needle 6. This way the upper straps 1 and 2 and the lower straps 3 and 4 can each be individually adjusted to properly fit over the tab 7 of upper needle assembly 9 and tab 8 of lower needle assembly 10, respectively. The needles 5 and 6 are held in place by crossing straps 1 and 2, and 3 and 4, over tabs 7 and 8, respectively. The needles are thereby securely fastened without adhesive bandages, the injection points and treatment sites are easily observed, and the patient has some ability for movement.

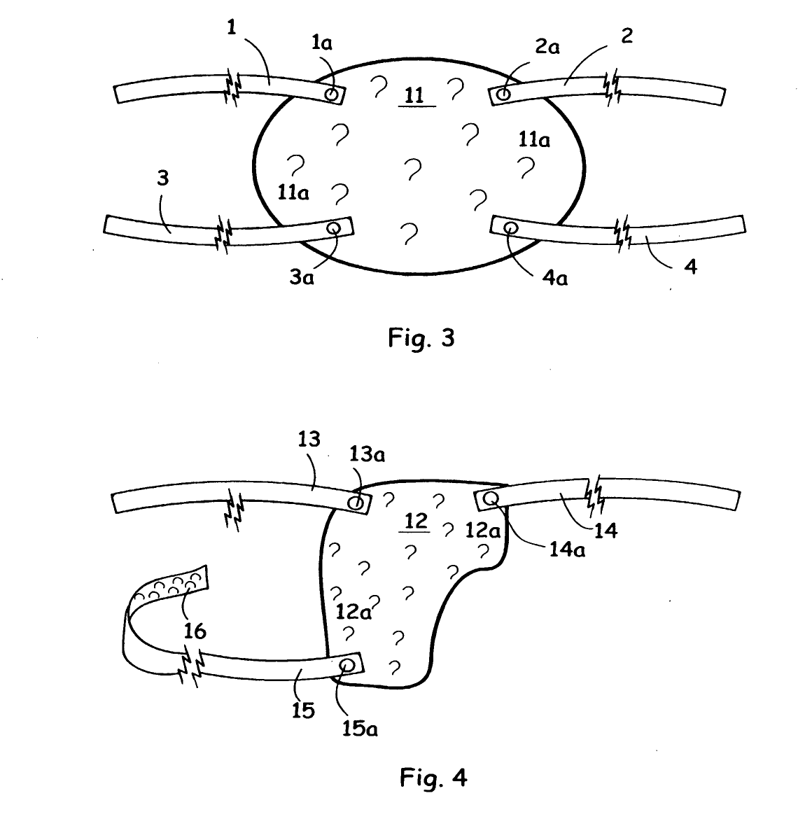

FIG. 3 shows the pad 11 of FIGS. 1 and 2 when it is not applied to a patient. The pad 11 has hooks 11a of the VELCRO™ system on an outer surface as indicated in FIG. 3. The four straps 1, 2, 3 and 4 are fastened to the pad 11 at 1a, 2a, 3a, and 4a by grommets that are applied in such a manner so that the straps are able to rotate into the direction by which they are forced by the application of the pad and the location of the needles themselves. Straps 1, 2, 3 and 4 have loops of the VELCRO™ system applied to the surface facing pad 11 (not shown), which also faces the patient when the device is applied. The loops of the VELCRO™ system tend to be softer and more compliant than the hooks. Thus patient comfort can be improved with this arrangement of the hooks and loops. Furthermore, the more compliant loops will better conform to the tabs 7 and 8 thereby providing for good retention thereof. Alternatively, the outer surface of the pad 11 could have the loops thereon so that the VELCRO™ system cannot snag on any outer garment. However, this arrangement would require the hooks to be applied to straps 1, 2, 3 and 4 and the previously mentioned patient comfort and tab retention may be lessened as, a result.

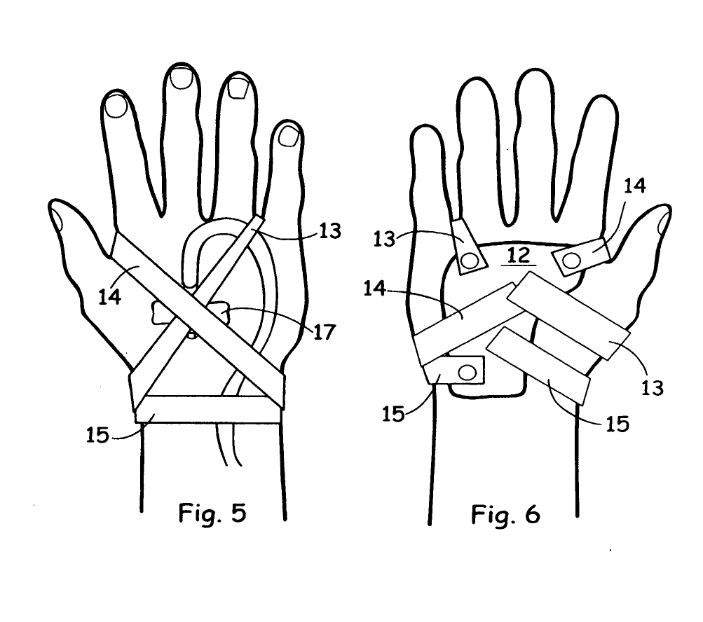

FIG. 4 illustrates a pad 12 having three corners in a triangular shape where straps 13, 14 and 15 are applied. Again, the straps 13, 14 and 15 are fastened by way of grommets 13a, 14a and 15a, respectively, in a rotational manner as was explained with regard to FIG. 3, and have VELCRO™ loops on the surface facing pad 12 as exemplified by section 16 of strap 15. Again, the outer surface of the pad comprises hooks 12a of VELCRO™ as explained above. The pad 12 of FIG. 4 is used in the event when a needle is applied to the back of a hand as shown in FIGS. 5 and 6. The three straps 13, 14 and 15 are applied as shown in FIG. 5 where the straps 13 and 14 cross each other over a tab 17 and the strap 15 secures the implement around the wrist of the patient. FIG. 6 shows the implement of FIG. 5 as it is applied to the inside of the hand. The triangular shape of the pad 12 can clearly be seen in FIGS. 4 and 6.

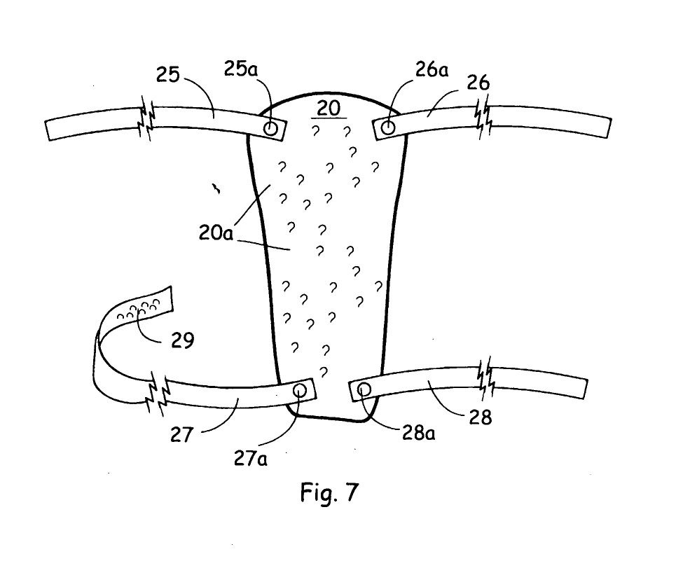

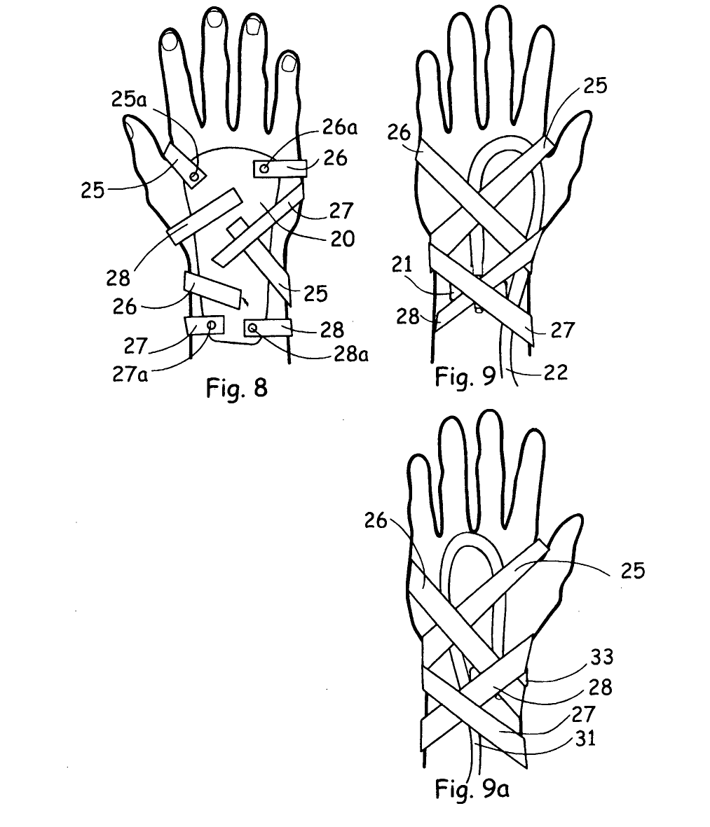

FIG. 7 shows a different pad 20 of a substantially rectangular configuration having straps 25, 26, 27 and 28 fastened by way of grommets 25a, 26a, 27a and 28a, respectively, in a rotational manner as discussed in pads 11 and 12, and have VELCRO™ loops on the surface facing pad 20 as exemplified by section 29 of strap 27. The outer surface of the pad 20 comprises hooks 20a of VELCRO™ as explained above. One end of pad 20 is somewhat enlarged where the grommets 25a and 26a are located. This pad can be used when a needle is inserted on the inside wrist of a hand as can be seen in FIGS. 8, 9 and 9a. Referring first to FIGS. 8 and 9, the pad 20 is placed on the outside of the hand and the straps 27 and 28 cross each other over a tab 21 of a needle assembly 22. Straps 25 and 26 serve to additionally secure the pad 20 and the tube of needle assembly 22 to the hand. Referring now to FIG. 9a, this system will work perfectly well even if the IV is somewhat offset from the middle of the wrist. The straps 26 and 28 cross over a tab 33 on an IV assembly 31. This is a somewhat complicated installation, but the cooperation between the pad 20 and the straps 25, 26, 27 and 28 makes for a versatile installation.

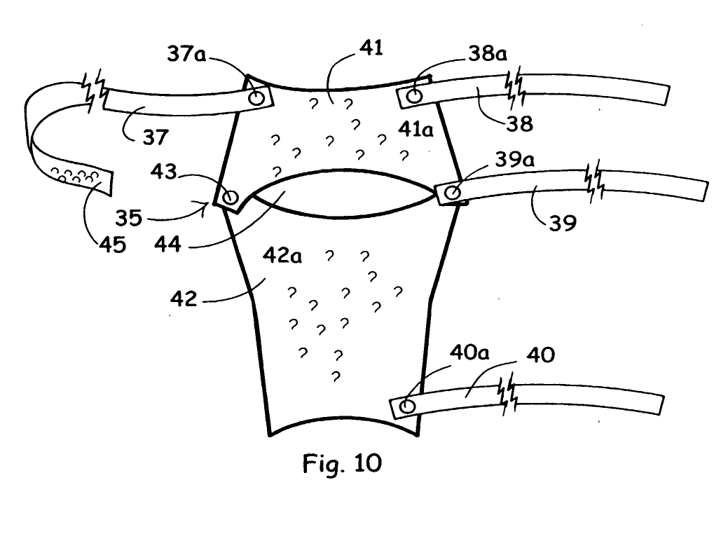

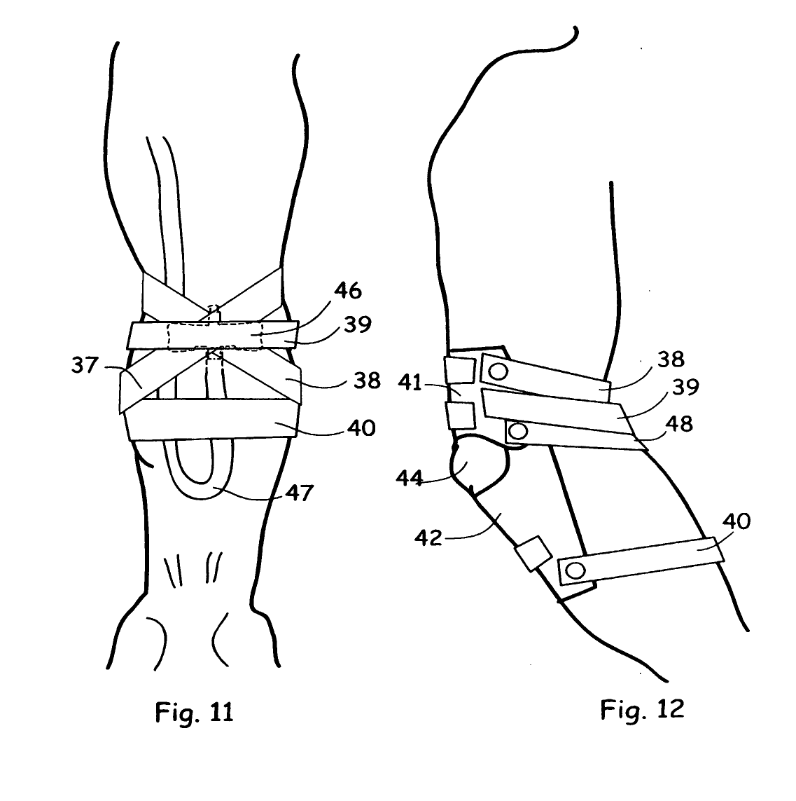

FIG. 10 shows a yet a different pad 35 which can be installed at the elbow of a patient. This pad comes is particularly useful when a needle is to be inserted at the inside of the inner arm opposite an elbow. Pad 35 has straps 37, 38, 39 and 40 fastened by way of grommets 37a, 38a, 39a and 40a, respectively, in a rotational manner as discussed in pads 11, 12 and 20, and have VELCRO™ loops on the surface facing pad 35 as exemplified by section 45 of strap 37. The pad 35 is made up of two parts 41 and 42 having outer surfaces comprising hooks 41a and 42a, respectively, of VELCRO™ as explained above. The parts 41 and 42 are connected by way of the grommets 43 and 39a. Grommet 39a is therefore fastening together strap 39 and parts 41 and 42. The two parts 41 and 42 have opposing curved inner edges and when connected together leave an open space 44 which will fit around the elbow. The grommets 43 and 39a enable the parts 41 and 42 to rotate around each other so that once the pad is installed on the elbow, the two parts 41 and 42 will be able to move with the movement of the elbow. FIG. 11 shows the installation of the pad 35. FIGS. 11 and 12 show how the pad 35 and an IV assembly 47 having a tab 46 are held in place. To this end, the strap 40 circles the arm below the elbow while holding a loop of tube of assembly 47 in place. The straps 37 and 38 cross each other over the tab 46 while the strap 39 circles around the arm just above the elbow. This arrangement assures for a secure holding of the IV needle in place while at the same time affording visibility of the performance of the needle itself while the arm may rotate around the elbow.

The pad itself, in all of the above described installations, should be made of a semi-rigid material. That is, it should not give in any planar direction; yet, it should be bondable so that it can conform to any contours of an arm, hand, leg or other part of the body. The pad should not turn or flex with the different directions the straps may take. The grommets should allow the straps to turn relative to the pad. The application of two straps crossing over the needle assembly and tab thereof as described in the above installations, enables a first strap to apply a first pressure to initially secure and position said assembly, and a second strap to apply a second pressure further securing said assembly. As noted above, the pad can comprise hooks of the VELCRO™ fastening system, thereby allow the softer loops thereof to be used on the straps which face the patient. Patient comfort can be improved with this arrangement of the hooks and loops. Furthermore, the more compliant loops will better conform to the tabs of needle assemblies thereby providing for good retention thereof. Alternatively, the outer surface of the pad 11 could have the loops thereon so that the VELCRO™ system cannot snag on any outer garment. However, this arrangement would require the hooks to be applied to the straps and the previously mentioned patient comfort and tab retention may be lessened as a result. In an alternative embodiment, adhesives could be used to attach the straps to the pads after wrapping said straps around the patient. In this embodiment, the adhesive could be applied such that it would not come into contact with the patient. This embodiment may be preferable when the device is to be disposed of after a single use. However, the VELCRO™ system may provide a more secure attachment of the straps to the pad than an adhesive. Furthermore, as noted above, the soft and compliant loops of the VELCRO™ system may provide for improved patient comfort and improved retention of the tabs of the needle assemblies.

As mentioned earlier, needles and cannulas are also referred to as venous access devices. The present invention can also be used with venous access device assemblies which do not have tabs, or as they are also called, wings or butterflies. Generally there will be a firm portion between the base of the needle or cannula and the tubing which is suitable for gripping or holding the venous access device assembly near the insertion point. A wing, butterfly, tab or said firm portion can be referred to as a stabilizing feature of the needle.

Given the benefit of the above disclosure of the present invention, those skilled in the art may envision how the present invention could be practiced using variations to the disclosed embodiments. For example, the present invention can be practiced and used to secure venous access devices to other locations of the body such as locations on the legs, and alternative pad shapes and strap arrangements can be envisioned, all without departing from the spirit and scope of the invention. Furthermore, the present invention can also be used to secure tubing and assemblies related to implanted venous catheters, which is yet another form of a venous access device. The foregoing detailed description should be regarded as illustrative rather than limiting and the appended claims, including all equivalents, are intended to define the scope of the invention.

What I claim is:

1. A device for securing a first venous access device comprising: a semi-rigid but flexible pad adapted to be installed on a part of a patient, said pad having flexible straps installed thereon, said straps having the capability of being separably fastened to said pad after surrounding said patient's part, said straps, when crossing each other in a first location which is opposite from said pad, can secure a stabilizing feature of said first venous access device while said straps continue around said patient's part to be fastened to said pad.

2. The device of claim 1, wherein said capability of fastening includes a hook and loop fastening system.

3. The device of claim 1, wherein said capability of fastening includes an adhesive fastening system.

4. The device of claim 1, wherein said pad has substantially the shape of a rectangle with said straps attached to each of the four corners.

5. The device of claim 1, wherein each of said straps are installed such that they may rotate relative to said pad.

6. The device of claim 1, wherein the device secures a second venous access device, including a first set of said straps securing said first venous access device and a second set of said straps securing said second venous access device where said straps of said first set cross each other and said straps of said second set cross each other.

7. The device of claim 1, wherein said pad is divided into two parts, each of said divided parts having curved edges facing each other to create an opening when connected together and are connected so as to be movable relative to each other.

8. The device of claim 1, wherein said pad has substantially the shape of a triangle with said straps attached to each of the three corners.

9. The device of claim 8, wherein said pad is placed in an inside of a hand, one of the straps crossing around a wrist of said hand and two of said straps continuing around a front of said hand in opposite directions to thereby cross a venous access device on an outside of said hand and continue around said hand to be connected to said pad.
